(19) United States
(12) Patent Application Publication
(10) Pub. No.: US 2026/0248511 A1
(43) Pub. Date: Aug. 27, 2026

(54) ALGORITHM FOR DETERMINING AND DISPLAYING COMPRESSION AND TISSUE GAP IN A CIRCULAR STAPLER

(71) Applicant: Cilag GmbH International, Zug (CH)

(72) Inventors: Matthew HILL, Cincinnati, OH (US); Evan STAMBLER, Cincinnati, OH (US); Mark OVERMYER, Cincinnati, OH (US)

(73) Assignee: Cilag GmbH International, Zug (CH)

(21) Appl. No.: 19/545,252

(22) Filed: Feb. 20, 2026

Related U.S. Application Data
(60) Provisional application No. 63/761,642, filed on Feb. 21, 2025.

(51) Int. Cl. A61B 17/115; A61B 17/00; A61B 90/00

(52) U.S. Cl. CPC A61B 17/1155; A61B 90/06; A61B 2017/00022; A61B 2017/00199; A61B 2017/00221; A61B 2090/066

(57) ABSTRACT

A circular stapler for cutting and applying one or more surgical staples to tissue is disclosed. The circular stapler includes various forms of detection systems that are configured to provide feedback to the surgeon that proper tissue compression has been achieved between an anvil of the circular stapler and a stapling head of the circular stapler before firing of the circular stapler. A surgical system includes a console and a controller communicatively coupled to the circular stapler which aids in processing and displaying data to provide feedback to the surgeon that proper tissue compression has been achieved between the anvil and the stapling head before firing of the circular stapler.

### CROSS REFERENCE TO RELATED APPLICATIONS

This application claims the benefit of U.S. Provisional Application No. 63/761,642, filed Feb. 21, 2025, which is incorporated by reference as if fully set forth.

### TECHNICAL FIELD

The present disclosure is directed to circular staplers for use in medical procedures, and more particularly to an algorithm for determining and displaying compression and tissue gap in a circular stapler used in medical procedures.

### BACKGROUND

In certain types of surgical procedures, the use of surgical staples has become the preferred method of joining tissue. Therefore, specially designed surgical staplers have been developed for these applications, which can be referred to as circular staplers in some examples. Circular staplers have become particularly useful for performing an anastomosis procedure, as is known. An anastomosis procedure includes joining sections of intestine of a patient together after a portion of intestine has been removed from the patient. The anastomosis procedure requires re-joining the ends of the two tubular sections together to form a continuous tubular pathway, which is accomplished by the circular stapler. During the surgical procedure, an anvil of the circular stapler must be properly connected to a trocar of the circular stapler to properly perform cutting and stapling of the tissue. Additionally, once the anvil is connected to the trocar, proper compression of the tissue and the tissue gap between the anvil and the stapling head is required to perform cutting and stapling of the tissue. If proper tissue compression and tissue gap are not achieved before firing (e.g., cutting and stapling), adverse outcomes and improperly secured tissue may occur. In existing circular staplers, it is difficult for the user (e.g., a surgeon) to determine if proper tissue compression and tissue gap has been achieved.

Therefore, there is a need to provide feedback to the user that proper tissue compression and tissue gap has been achieved before closing and firing of the circular stapler.

### SUMMARY OF THE DISCLOSURE

According to one aspect, a method of determining and displaying tissue compression in a circular stapler is provided. The method can include the steps of: establishing a range of expected compression values based on known tissue properties; gathering a tissue compression value for tissue compressed between an anvil of the circular stapler and a stapling head of the circular stapler; comparing the gathered tissue compression value to the range of expected compression values; and providing an indication that the gathered tissue compression value is within the range of expected compression values.

In one aspect, the range of expected compression values is established based on test data indicating a maximum tissue compression and a minimum tissue compression.

In one aspect, the maximum tissue compression is a maximum burst pressure of the tissue, and the minimum tissue compression is a minimum burst pressure of the tissue.

In one aspect, the known tissue properties comprises tissue thickness.

In one aspect, the known tissue properties comprises a position along a gastrointestinal tract.

In one aspect, the step of gathering the tissue compression value is achieved by at least one of a torque sensor and a force sensor.

In one aspect, the torque sensor is positioned within an adjustment knob of the circular stapler.

In one aspect, the step of comparing the gathered tissue compression value and the range of expected compression values is accomplished within at least one of a console and a controller communicatively coupled to the circular stapler.

In one aspect, the console and the controller are wirelessly communicatively coupled to the circular stapler, and the console and the controller are positioned remotely from the circular stapler.

In one aspect, the step of providing the indication that the gathered tissue compression value is within the range of expected compression values is provided on a display of the circular stapler.

In one aspect, providing the indication that the gathered tissue compression value is within the range of expected compression values is provided on a display of a console that is communicatively coupled to the circular stapler.

In one aspect, the step of providing the indication that the gathered tissue compression value is within the range of expected compression values is provided on a display of a controller that is communicatively coupled to the circular stapler.

In one aspect, a further step can include providing an indication of over-compression or under-compression on a display of the circular stapler.

In one aspect, a further step can include providing an indication of staple height on the display of the circular stapler.

In one aspect, the indication of over-compression or under-compression is provided on a display of a console that is communicatively coupled to the circular stapler.

In one aspect, the indication of staple height is provided on the display of the console.

In one aspect, the indication of over-compression or under-compression is provided on a display of a controller that is communicatively coupled to the circular stapler.

In one aspect, the indication of staple height is provided on the display of the controller.

According to another aspect, a circular stapler apparatus is provided. The circular stapler apparatus can include an anvil, a stapling head arranged to cooperate with the anvil to compress tissue therebetween, and an adjustment mechanism operable to move the anvil relative to the stapling head to compress the tissue. The circular stapler apparatus can further include a sensor configured to provide an output indicative of a tissue compression value for the tissue compressed between the anvil and the stapling head, a display, and a controller in communication with the sensor and the display. The controller can include at least one processor and memory storing instructions that, when executed by the at least one processor, cause the controller to: establish a range of expected compression values based on known tissue properties; determine, based on the output of the sensor, the tissue compression value; compare the tissue compression value to the range of expected compression values; and cause the display to present an indication that the tissue compression value is within the range of expected compression values.

In one aspect, the sensor can include a torque sensor positioned within an adjustment knob of the circular stapler apparatus and configured to provide the output indicative of the tissue compression value based on torque applied to the adjustment knob.

### BRIEF DESCRIPTION OF THE DRAWINGS

The foregoing Summary as well as the following Detailed Description will be best understood when read in conjunction with the appended drawings, which illustrate exemplary embodiments. In the drawings:

FIG. 1 is a perspective view of an exemplary embodiment of a circular stapler according to the present disclosure.

FIG. 2 is a partial side-view of the circular stapler of FIG. 1.

FIG. 3 is a magnified perspective view of a distal end of the circular stapler of FIG. 1, illustrating an anvil separated from a trocar of the circular stapler.

FIG. 4 is a magnified perspective view of a proximal end of the circular stapler of FIG. 1, illustrating an adjustment knob of the circular stapler.

FIG. 5 is a schematic perspective view of a surgical system according to the present disclosure.

FIG. 6 is a magnified side view illustrating thin tissue compressed between the anvil and a stapling head of the circular stapler illustrated in FIG. 3.

FIG. 7 is a magnified side view illustrating thick tissue compressed between the anvil and a stapling head of the circular stapler illustrated in FIG. 3.

FIG. 8A is a graphical representation of limits of compression based on testing data.

FIG. 8B is a schematic diagram of a display that shows the limits of compression to a user during use of the circular stapler.

FIG. 9A is another graphical representation of limits of compression based on testing data.

FIG. 9B is another schematic diagram of a display that shows the limits of compression to a user during use of the circular stapler.

FIG. 9C is a chart illustrating the correlation between tissue thickness and staple height, corresponding to the graphical representation of FIG. 9A.

FIG. 10A is another schematic diagram of a display that shows the limits of compression to a user during use of the circular stapler, as well as previous compression values.

FIG. 10B is another schematic diagram of a display that shows the limits of compression to a user during use of the circular stapler, including incremental progress indicators.

FIG. 11A is another schematic diagram of a display that shows limit warnings for compression to a user during use of the circular stapler.

FIG. 11B is a graphical representation of the limits of compression based on testing data, for the display of FIG. 11A.

FIG. 11C is another schematic diagram of a display that shows limit warnings for compression to a user during use of the circular stapler.

FIG. 11D is a graphical representation of the limits of compression based on testing data, for the display of FIG. 11C.

FIG. 12A is a graphical representation of tissue compression/knob torque versus the staple height during compression of the tissue.

FIG. 12B is a schematic diagram of a display that shows the limits of compression to a user during use of the circular stapler.

FIG. 12C is a graphical representation of an algorithm that smooths the curve of tissue compression/knob torque versus the staple height during compression of the tissue.

FIG. 13 is a graphical representation of a process for implementing a pre-compression timer according to the present disclosure.

FIG. 14 is a schematic representation of a process implementing a pre-compression timer according to the present disclosure.

### DETAILED DESCRIPTION

Certain terminology is used in the following description for convenience only and is not limiting. The words “front”, “rear”, “upper”, and “lower” designate directions in the drawings to which reference is made. The words “inwardly” and “outwardly” refer to directions towards and away from parts referenced in the drawings. “Axially” refers to a direction along the axis of a shaft or other cylindrically shaped component. A reference to a list of items that are cited as “at least one of a, b, or c” (where a, b, and c represent the items being listed) means any single one of the items a, b, or c, or combinations thereof. The terms “generally” and “approximately” are to be construed as within 10% of a stated value or ratio, unless otherwise noted. Additionally, the terms “proximal” and “distal” are used with reference to a handle portion of a circular stapler disclosed herein. The term “proximal” referring to the portion closest to the handle portion and the term “distal” referring to the portion located away from the handle portion in a direction of a tip of the circular stapler. The terminology includes the words specifically noted above, derivatives thereof, and words of similar import.

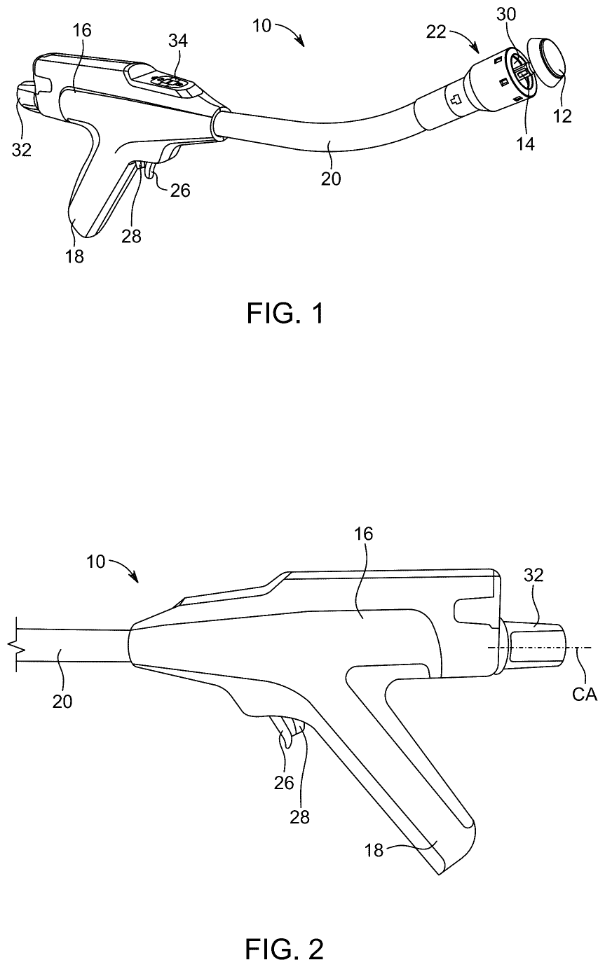

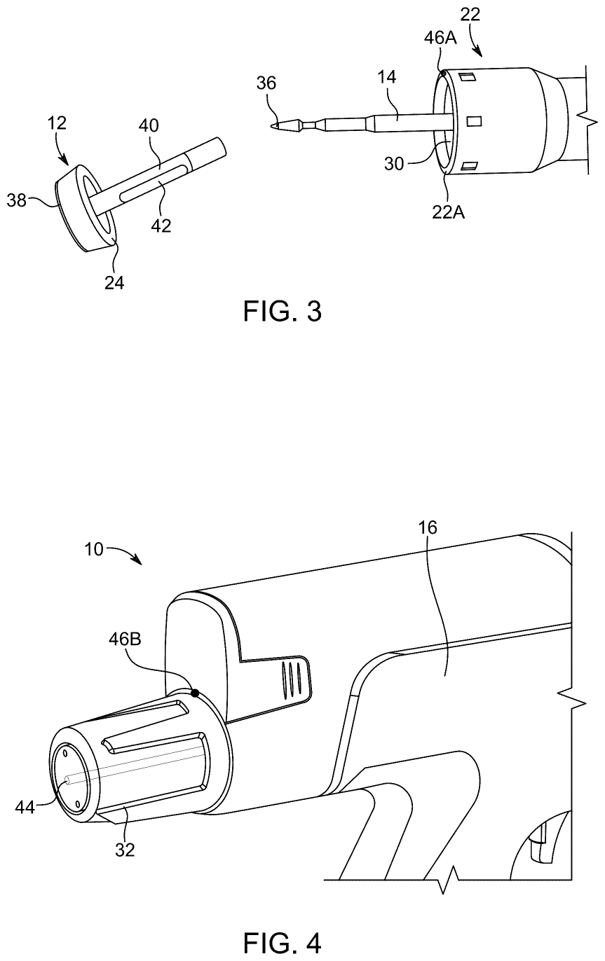

FIG. 1 is a perspective view of an exemplary embodiment of a circular stapler 10 according to the present disclosure. FIG. 2 is a partial side-view of the circular stapler 10. FIG. 3 is a magnified perspective view of a distal end of the circular stapler 10, illustrating an anvil 12 separated from a trocar 14 of the circular stapler 10. FIG. 4 is a magnified perspective view of a proximal end of the circular stapler 10, illustrating an adjustment knob 32 of the circular stapler 10. FIGS. 1-4 will be discussed together. The circular stapler 10 includes a housing 16 with a handle 18 extending downwards and away from the housing 16. The handle 18 may be grasped by a user (e.g., a surgeon) while using the circular stapler 10. It is to be understood that the terms “user” and “surgeon” may be used interchangeably throughout the present disclosure, with the intended meaning being the person holding and operating the circular stapler 10. Additionally, it is to be understood that the “user” could be any person that is applicable for the purpose of this disclosure. In other embodiments, the circular stapler 10 can be removably coupled to or integrated into a robotics system in which a robotic arm supports and operates the circular stapler 10 based on the surgeon's instructions/guidance through a remotely operated graphical user interface (GUI) or other controller communicatively coupled to the robotic arm/system.

A shaft 20 extends outwards from the housing 16, with the shaft 20 having the shape of an elongated tube with a circular cross-section. Additionally, the shaft 20 may include a slight bend or curvature, such that the shaft 20 is not a straight tube. A stapling head 22 is positioned at a distal end of the shaft 20, with the distal end of the shaft 20 being the end furthest from the housing 16 of the circular stapler 10. A trocar 14 is positioned within and axially aligned with the stapling head 22, and the trocar 14 is axially translatable relative to the stapling head 22. In other words, the trocar 14 can translate axially inwardly and outwardly relative to the stapling head 22, such that the trocar 14 can extend axially outwards of the stapling head 22 (see FIG. 3) and the trocar 14 can be positioned fully within and surrounded by the stapling head 22. An anvil 12 is removably coupled to the trocar 14, such that the anvil 12 can be connected to and removed from the trocar 14, discussed further below. When the anvil 12 is coupled to the trocar 14, the anvil 12 is axially aligned with the trocar 14 and therefore also axially aligned with the stapling head 22. Additionally, when the anvil 12 is coupled to the trocar 14, the anvil 12 is also axially translatable relative to the stapling head 22. The anvil 12 includes a staple forming surface 24 that is configured to engage with a distal face or surface of the stapling head 22, discussed further below.

A trigger 26 is pivotally coupled to the housing 16 adjacent the handle 18, with the trigger 26 being pivotal relative to the housing 16. Actuation of the trigger 26 causes firing or operation of the circular stapler 10, discussed further below. A safety 28 is coupled to the housing 16 and positioned adjacent the trigger 26, between the trigger 26 and the handle 18. The safety 28 is pivotal relative to the housing 16, and the safety 28 is configured to prevent the trigger 26 from being actuated when in the locked or safe position, and the safety allows the trigger 26 to be actuated when in the released or fire position. The released or fire position being the position in which the safety 28 is pivoted away from the trigger 26, allowing the trigger 26 to be actuated which fires or operates the circular stapler 10. When the trigger 26 is actuated, an internal drive system (not shown) of the circular stapler 10 operates within the shaft 20 to cause staples to be dispensed from the stapling head 22 into forming contact with the staple forming surface 24. Simultaneously, a knife 30 (see FIG. 3), that is operably supported within the stapling head 22, translates axially outwards from the stapling head 22 to cut tissue held within a circumference of the stapling head 22.

As shown best in FIGS. 2 and 4, the circular stapler 10 also includes an adjustment knob 32 coupled to a proximal end of the housing 16 that is furthest from the stapling head 22 of the circular stapler 10. The adjustment knob 32 is configured to rotate both clockwise and counterclockwise about a central axis CA of the adjustment knob 32. The adjustment knob 32 is connected to the internal drive system (not shown in FIGS. 1-4) within the housing 16 of the circular stapler 10, and the distal end of the internal drive system is connected to the trocar 14 adjacent and within the stapling head 22. Rotation of the adjustment knob 32 causes actuation of the internal drive system within the housing 16 and the shaft 20, which in turn causes the trocar 14 to translate axially inwardly and outwardly relative to the stapling head 22. As such, the user rotates the adjustment knob 32 at a proximal end of the circular stapler 10 to control the axial translation and positioning of the trocar 14 relative to the stapling head 22 at the distal end of the circular stapler 10. The circular stapler 10 can also include a display 34 that is positioned on a side of the housing 16, opposite the extending direction of the handle 18. The display 34 provides an indication when the tissue compressed between the anvil 12 and the stapling head 22 has reached an appropriate tissue compression, discussed further below.

Referring to FIG. 3, the knife 30 is a circular shaped knife 30 that is positioned within and axially aligned with the stapling head 22. When the trigger 26 is pulled and the internal drive mechanism is actuated (or fired), the knife 30 quickly translates axially outwards from the stapling head 22 and then retracts back into the stapling head 22. The knife 30 is configured to cut tissue that is compressed between the staple forming surface 24 of the anvil 12 and the distal end face 22A of the stapling head 22. The trocar 14 is an elongated metallic shaft with a sharp-pointed distal tip 36 that is configured to cut through tissue. It is to be understood that the trocar 14 can be any surgical instrument with cutting edges at the distal tip 36 for cutting tissue. Additionally, the trocar 14 is shaped and sized to couple to the anvil 12.

The anvil 12 includes an anvil shroud 38 which is a generally circular body portion of the anvil 12. The staple forming surface 24 is formed on the underside of the anvil shroud 38, which underside faces the stapling head 22 when the anvil 12 is coupled to the trocar 14. An anvil shaft 40 extends from the underside of the anvil shroud 38, in a direction axially away from the anvil shroud 38 such that the anvil shaft 40 is axially aligned with the anvil shroud 38. The anvil shaft 40 can have the shape of a hollow or a partially hollow elongated cylinder, which shape is a female mating shape to the male mating shape of the trocar 14. Therefore, the anvil shaft 40 and anvil 12 include complimentary shapes and sizes, such that the anvil 12 can be inserted over the distal tip 36 of the trocar 14 to be coupled to and/or removed from the trocar 14. The anvil 12 can further include at least one retaining clip 42 pivotally coupled to the anvil shaft 40. In some examples, the at least one retaining clip 42 can be a leaf-type spring or other spring component that snaps or latches onto features of the trocar 14 to retain the anvil 12 on the trocar 14. Further, in some examples, the at least one retaining clip 42 can be two retaining clips 42 positioned on opposite sides of the anvil shaft 40.

As discussed, the circular stapler 10 includes the elongated shaft 20 and a distal stapling head 22 with a stapling mechanism mounted to the distal end of the shaft 20. The stapling head 22 can also include a stapling cartridge (not shown) that contains a plurality of staples configured in a concentric circular array. The knife 30 can be concentrically mounted within the stapling cartridge and configured to travel axially within the stapling cartridge. Additionally, the trocar 14 can extend axially from a center of the stapling cartridge, and the trocar 14 is movable relative to the stapling cartridge. The trocar 14 is also adapted to be removably coupled to the anvil 12, as previously discussed. The anvil 12 is configured to form the ends of the staples as they are driven into the staple forming surface 24 of the anvil 12. The distance between a distal face of the staple cartridge and the staple forming surface 24 of the anvil 12 is controlled by rotating the adjustment knob 32 mounted to the proximal end of the housing 16 of the circular stapler 10. The adjustment knob 32 being configured for controlling the axial movement of the trocar 14. Tissue clamped between the distal end face 22A of the stapling head 22 and the staple forming surface 24 of the anvil 12 is simultaneously stapled and cut when the trigger is actuated by the surgeon.

In some examples, when performing an anastomosis using the circular stapler 10, the intestinal tissue is stapled using double rows of staples being placed on either side of the tissue of the intestine to be removed. The adjoining sections of tissue are simultaneously cut as the adjoining sections of tissue are stapled. In such examples, the surgeon typically inserts the anvil 12 into the proximal end of the lumen (intestinal tissue), proximal of the staple line. This is done by inserting the anvil shroud 38 into an entry port cut into the proximal lumen by the surgeon. In some instances, the anvil 12 can be placed transanally, by placing the anvil shroud 38 on the distal end of the circular stapler 10 and inserting the instrument through the rectum. The surgeon then ties the proximal end of the intestine to the anvil shaft 40 using a suture or other conventional tying device.

Next, the surgeon may cut excess tissue adjacent to the tie and the surgeon attaches the anvil 12 to the trocar 14 of the circular stapler 10. The surgeon then closes the gap between the anvil 12 and stapling head 22 by rotating the adjustment knob 32, thereby retracting the trocar 14 with the attached anvil 12 towards the distal end face 22A of the stapling head. The anvil 12 and trocar 14 are retracted far enough such that the proximal and distal ends of the intestine are clamped in the gap between the staple forming surface 24 and the distal end face 22A of the stapling head 22. During closing of the gap, the surgeon watches the display 34, which indicates when the correct amount of compression has been achieved based on the tissue thickness, among other variables. When the correct compression has been achieved, the surgeon actuates the trigger 26 causing several rows of staples to be driven through both ends of the intestine and formed, thereby joining the ends and forming a tubular pathway. Simultaneously, as the staples are driven and formed, the knife 30 is driven through the intestinal tissue ends, cutting the ends adjacent to the inner row of staples. The surgeon then withdraws the circular stapler 10 from the intestine and the anastomosis procedure is complete.

During the aforementioned stapling and cutting process, it is often difficult for the surgeon to determine if proper tissue compression has been achieved between the distal end face 22A of the stapling head 22 and the staple forming surface 24 of the anvil 12. If proper tissue compression is not achieved before the cutting and stapling, adverse outcomes and improperly secured tissue may occur. In previous circular staplers, it was difficult for the user (e.g., surgeon) to determine if proper tissue compression has been achieved. Circular stapler 10 and a surgical system 50 alleviate the aforementioned issues by providing feedback to the user that proper tissue compression has been achieved before the cutting and stapling by the circular stapler 10, described in detail below.

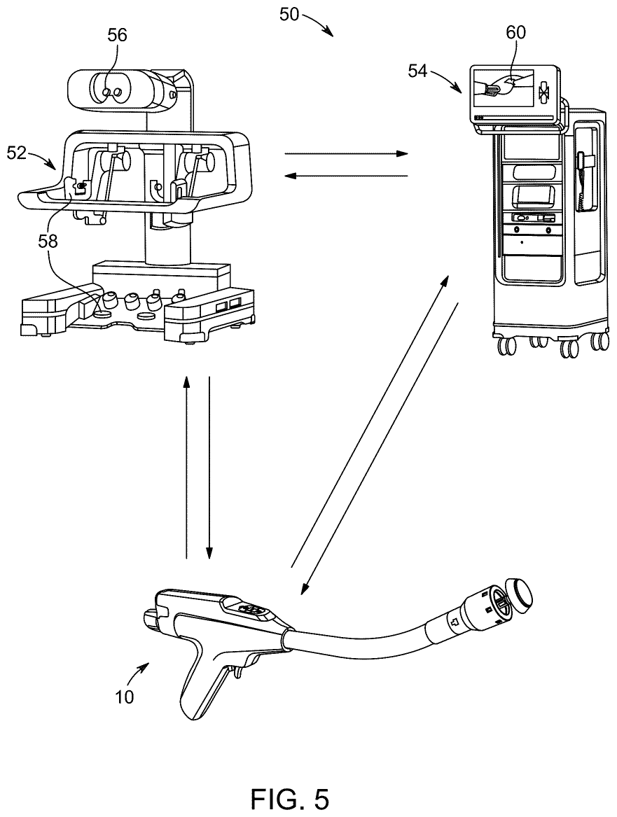

FIG. 5 is a schematic perspective view of an example of a surgical system 50 according to the present disclosure. The surgical system 50 includes the circular stapler 10 which is communicatively coupled to both a console 52 and a controller 54. In some examples, the circular stapler 10 is communicatively coupled to the console 52 and the controller 54 through a wireless connection, such as for example through Bluetooth or WiFi connections, among other options not specifically listed. In other examples, the circular stapler 10 is communicatively coupled to the console 52 and the controller 54 through a wired connection using electrical wires (not shown). In either example, the circular stapler 10 is configured to transfer data between the circular stapler 10 and the console 52 and between the circular stapler 10 and the controller 54. Additionally, it is to be understood that the console 52 and the controller 54 are communicatively coupled such that data can also be transferred between the console 52 and the controller 54, through either a wireless or wired connection.

The console 52 is configured to control and operate certain functionality of the circular stapler 10 through commands sent from the console 52 to the circular stapler 10. Specifically, an assistant can hold the circular stapler 10 at the location of the patient while the surgeon (or other user) is positioned remotely (within the same room) from the circular stapler 10 at the console 52. The surgeon can then interact with the console 52 to cause the commands to be sent from the console 52 to the circular stapler 10. For example, the surgeon can manipulate the console 52 to cause the trocar 14 with the attached anvil 12 to retract and translate towards the stapling head 22 to compress tissue between the stapling head 22 and the anvil 12, as previously discussed. The console 52 can include a console display 56 and a plurality of touchpoints 58. The console display 56 can be communicatively coupled to at least one camera (not shown) that is positioned adjacent the area the surgical procedure is being performed on the patient. Therefore, the surgeon can view the console display 56 to view the image being transferred from the at least one camera to the console 52. The plurality of touchpoints 58 can include at least one of foot pedals and hand operated levers or controls. As such, the plurality of touchpoints 58 are the components the surgeon actuates and operates to send the command signals to the circular stapler 10.

The controller 54 can be positioned remotely from both the circular stapler 10 and the console 52, and the controller 54 can be communicatively coupled to both the circular stapler 10 and the console 52 for data transfer between the components. The controller 54 is configured to process data received from both the circular stapler 10 and the console 52. As such, it is to be understood that the controller 54 includes (although not shown) internal processors, memories, and stored algorithms that are configured for performing the desired data processing and analysis to determine that proper tissue compression has been achieved, among other capabilities, discussed further below. The controller 54 can include a controller display 60 that can be configured to display images provided by cameras, as previously discussed, as well as other data related to the circular stapling process. In some examples, the controller display 60 can display the anvil 12 opening progress feedback, a firing status (i.e., firing complete) feedback, a compression time feedback, the anvil 12 attachment state feedback, a firing initiation feedback, a spike hidden feedback, a full spike extension feedback, and a communication connection status feedback, among other information not specifically listed. Therefore, the controller 54 is configured to aid in the surgery by processing and displaying data relevant to the circular stapling surgical procedure.

The present disclosure includes embodiments where the circular stapler 10 is used with surgical system 50 (described above) and embodiments with the circular stapler is operated manually in a stand-alone manner by a user.

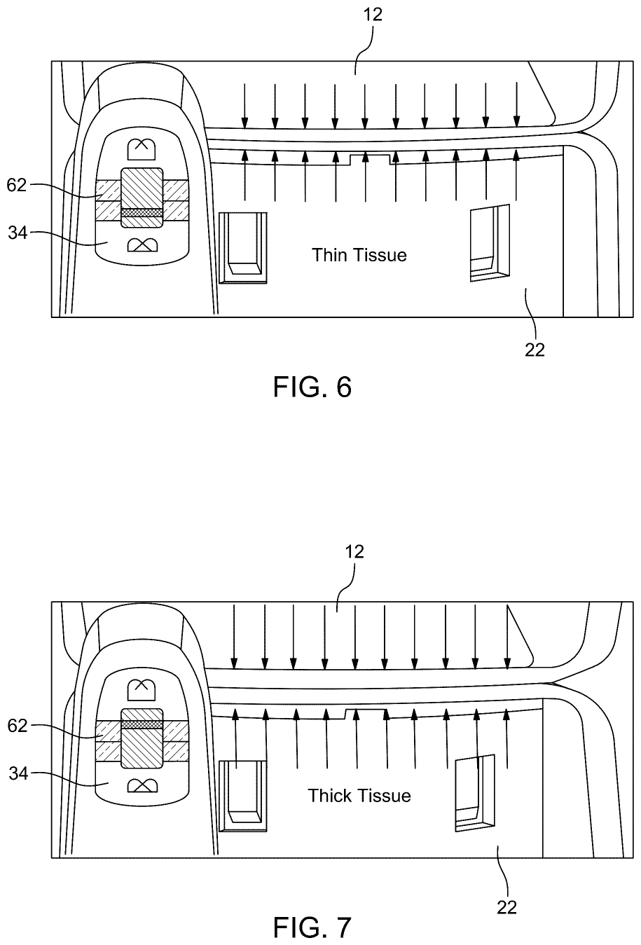

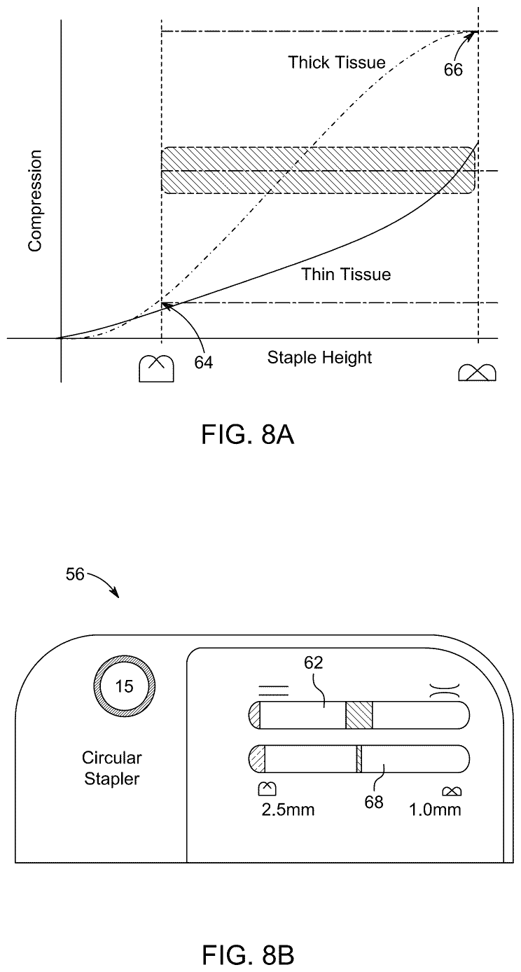

FIG. 6 is a magnified side view illustrating thin tissue compressed between the anvil 12 and the stapling head 22 of the circular stapler 10. FIG. 7 is a magnified side view illustrating thick tissue compressed between the anvil 12 and the stapling head 22 of the circular stapler 10. FIG. 8A is a graphical representation of limits of compression based on testing data. FIG. 8B is a schematic diagram of the console display 56 that shows the limits of compression to a user during use of the circular stapler 10. FIGS. 6-8B will be discussed together, and FIGS. 6-8B overall illustrate testing based compression scale development for the circular stapler 10.

Referring to FIG. 6, the anvil 12 and the stapling head 22 are illustrated clamping and compressing thin tissue. Further, FIG. 6 also schematically illustrates the display 34 that is positioned on the circular stapler 10 (see FIG. 1), but it is to be understood that the display 34 is not positioned adjacent the anvil 12 or the stapling head 22 as schematically illustrated in FIG. 6. The display 34 can include a compression zone 62, which is a scale on the display 34 that indicates when proper tissue compression has been achieved. The compression zone 62 includes an upper limit and a lower limit, which are based on previous testing data and calibration of the circular stapler 10. As illustrated in FIG. 6, when thin tissue is compressed between the anvil 12 and the stapling head 22, proper tissue compression is achieved closer to the lower limit of the compression zone 62. Referring to FIG. 7, when thick tissue is compressed between the anvil 12 and the stapling head 22, proper tissue compression is achieved closer to the upper limit of the compression zone 62. As such, when a user knows that the tissue being compressed is thin or thick, the user knows that proper tissue compression is achieved closer to the lower limit or closer to the upper limit of the compression zone 62, respectively. This aids the user in ensuring proper tissue compression is achieved before the stapling and cutting processes. Proper tissue compression is a critical aspect to ensuring the staples correctly dispense, form, close, and seal the ends of the tissue together.

As discussed, the user needs to know when proper tissue compression has been achieved and also if there is full contact and appropriate distances set between the anvil 12 and stapling head 22 before firing of the circular stapler 10. The compression zone 62 that indicates when proper tissue compression has been achieved, based on thin or thick tissue, was developed based on previous testing data, such as for example previous testing using porcine tissue. Specifically, the adjustment knob 32 can include a torque sensor 44 (see FIG. 4) and the torque on the adjustment knob 32 can be monitored during the testing to develop a tissue compression scale (compression zone 62) that is communicated to the user. More specifically, the torque recorded by the torque sensor 44 during porcine tissue compression was evaluated at various adjustment knob 32 torque values using histology and staining to identify under or over compression. The testing data was used to develop a compression scale which correlates the torque on the adjustment knob 32 to the tissue compression between the anvil 12 and the stapling head 22, based on the thickness of the tissue being compressed.

In other examples, a force sensor 46A (see FIG. 3) can be positioned at the distal end face 22A of the stapling head 22, and the force sensor 46A can detect the compression force between the anvil 12 and the stapling head 22. In yet other examples, a force sensor 46B (see FIG. 4) can be positioned between the housing 16 and the adjustment knob 32, and the force sensor 46B can be correlated to the compression force between the anvil 12 and the stapling head 22, such that the compression force at the force sensor 46B indicates a certain compression force between the housing 16 and the adjustment knob 32, which is based on previous testing data and calibration of the circular stapler 10. In further examples, the circular stapler 10 can include various sensors positioned throughout the device that are configured to detect torque or other forces which can be correlated to the compression force between the anvil 12 and the stapling head 22, as will be appreciated by those having skill in the art.

FIG. 8A is a graphical representation of the limits of tissue compression based on the testing data for both thin and thick porcine colon tissue. FIG. 8B illustrates the console display 56 that shows the limits of compression to a user during use of the circular stapler 10. As illustrated in the graph of FIG. 8A, the tissue compression is compared to the staple height that will result based on thin or thick tissue. On the X-axis, the staple height is higher on the left and decreases to the right (further from the Y-axis). For example, for thin tissue, if the compression is lower the staple height will be greater due to the increased gap size between the anvil 12 and the stapling head 22. Likewise, for thin tissue, if the compression is higher the staple height will be smaller due to the decreased gap size between the anvil 12 and the stapling head 22. The aforementioned correlation between compression and staple height also applies for thick tissues, as illustrated in the graph of FIG. 8A.

The testing results were used to determine the minimum tissue compression 64 for thin tissue that is required for acceptable burst pressures of the stapled tissue, and the testing results were used to determine the maximum tissue compression 66 that can be used for thick tissue and still achieve acceptable burst pressures for the stapled tissue. This information was then used to set the upper and lower limits of the compression zone 62, which is shown on the console display 56 for the user to monitor during the stapling process. As such, the user can interact with the console 52 to cause the anvil 12 to translate towards the stapling head 22 to compress the tissue, while the user monitors the tissue compression on the console display 56. The user has knowledge whether the tissue being stapled is thin or thick, based on visual and/or tactile inspection of the tissue, and therefore the user can determine when proper tissue compression is achieved based on the compression zone 62 displayed on the console display 56. As illustrated, the console display 56 can also indicate to the user the staple height 68 that will result during the stapling process. Once proper tissue compression has been achieved, the user can then fire the circular stapler 10 to perform the cutting and stapling processes.

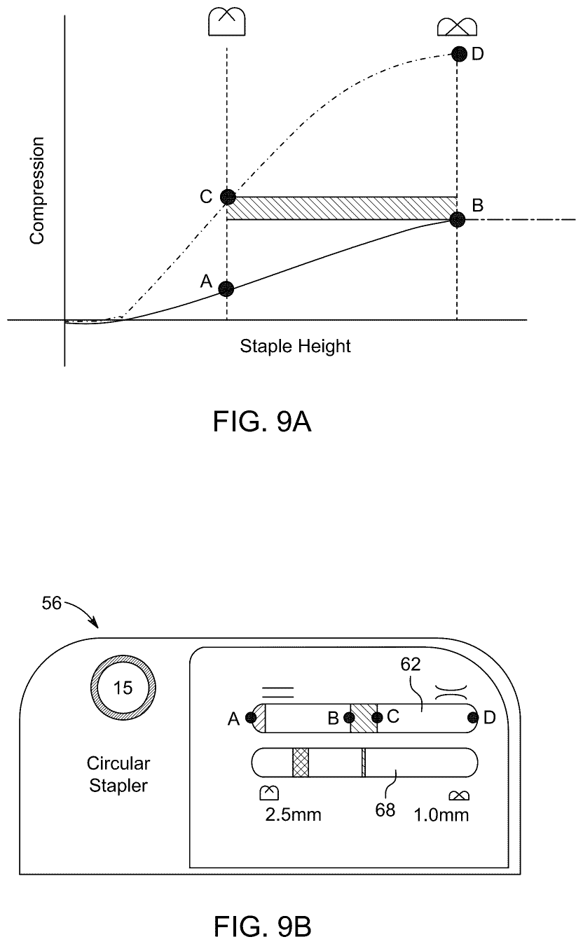

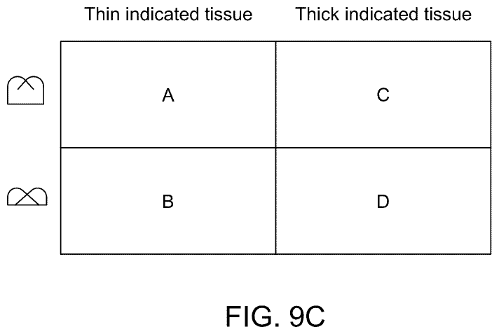

FIG. 9A is another graphical representation of limits of tissue compression based on testing data. FIG. 9B is another schematic diagram of the console display 56 that shows the limits of tissue compression to a user during use of the circular stapler 10. FIGS. 9A-9B are substantially similar to FIGS. 8A-8B, respectively, and therefore the disclosure regarding FIGS. 8A-8B equally applies to FIGS. 9A-9B unless otherwise noted.

FIG. 9A illustrates tissue compression points A and B which correspond to the minimum and maximum, respectively, tissue compression for thin tissue, which results in proper tissue compression before firing of the circular stapler 10, based on test data. FIG. 9A also illustrates tissue compression points C and D which correspond to the minimum and maximum, respectively, tissue compression for thick tissue, which results in proper tissue compression before firing of the circular stapler 10, based on test data. As illustrated in FIG. 9B, the tissue compression points A, B, C, and D are linked to the compression zone 62 that is displayed on the console display 56 to provide the user reference points for compression depending on the tissue thickness. Specifically, tissue compression point A corresponds to the minimum required compression for thin tissue, and tissue compression point B corresponds to the maximum compression for thin tissue, for properly securing the tissue together. Likewise, tissue compression point C corresponds to the minimum required compression for thick tissue, and tissue compression point D corresponds to the maximum compression for thick tissue, for properly securing the tissue together.

During surgery, the user has knowledge whether the tissue being stapled is thin or thick, and therefore the user can determine when proper tissue compression is achieved based on the compression zone 62 displayed on the console display 56. As illustrated, the console display 56 can also indicate to the user the staple height 68 that will result during the stapling process. Once proper tissue compression has been achieved, the user can then fire the circular stapler 10 to perform the cutting and stapling processes. FIG. 9C is a chart illustrating the correlation between tissue thickness and staple height, corresponding to the graphical representation of FIG. 9A. Specifically, thin and thick tissue experiencing less compression will result in greater staple heights, and thin and thick tissue experiencing greater compression will result in smaller staple heights, as previously discussed. Further, FIG. 9C illustrates how the staple height corresponds to the tissue compression points A, B, C, and D of FIGS. 9A-9B.

Table 1: Tissue Thickness Examples, below, indicates the relative ranges for what is considered thin and thick tissue based the position along the gastrointestinal (G.I.) tract, with the lower G.I. tract comprising the distance from the colon to the rectum, and the upper G.I. tract comprising the distance from the esophagus to the stomach. It is to be understood that the below table is not to be limiting and all-inclusive, but rather provides example thin and thick tissue values depending on the position along the G.I. tract.

TABLE 1
Tissue Thickness Examples

| | Thin Indicated Tissue | Thick Indicated Tissue | Design Limit Tissue |
|---|---|---|---|
| Lower GI | Jejunum to Jejunum | *Colon (0.6-1.3 mm) to | *Colon (0.6-1.3 mm) to |
| (CDH31P & | Total measured thickness | Rectum/Colon (1.1- | Rectum/Colon (1.4- |
| CDH29P) | 1.0 +/− 0.3 mm | 1.7 mm) | 2.3 mm) |
| | | Total additive thickness of | Total additive thickness of |
| | | 2.2 mm to 2.7 mm | 2.8 mm to 3.3 mm |
| Upper GI | | Esophagus (0.7-1.3 mm) to | Esophagus (1.0-1.5 mm) to |
| (CDH25P & | | Stomach (1.1-1.7 mm) | Stomach (1.4-1.9 mm) |
| CDH23P) | | Total additive thickness of | Total additive thickness of |
| | | 2.2 mm to 2.7 mm | 2.8 mm to 3.3 mm |

*The use of rectum and colon interchangeably to achieve the appropriate tissue thickness was discussed with medical affairs and accepted as an appropriate model. If there is difficulty in achieving the total thickness with two pieces of rectum/colon tissue it is acceptable to use up to three layers that are additive to the total thickness specified.

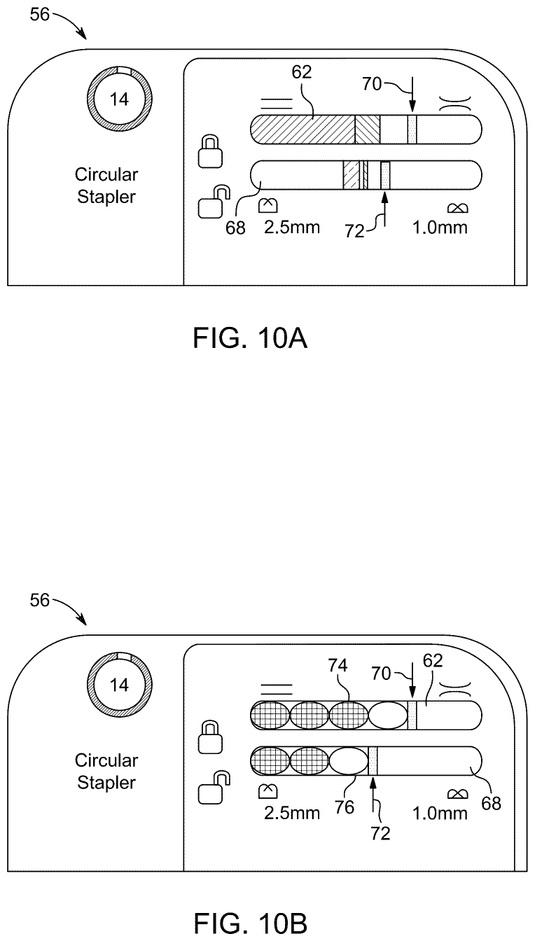

FIG. 10A is another schematic diagram of the console display 56 that shows the limits of tissue compression to a user during use of the circular stapler 10. Further, in the embodiment of the console display 56 illustrated in FIG. 10A, the console display 56 also displays the previous compression values as references points for the user. As discussed, the user needs to know when proper tissue compression has been achieved and also if there is full contact and appropriate distances set between the anvil 12 and stapling head 22 before firing of the circular stapler 10. In the illustrated embodiment, data related to a final previous compression value 70 and a final previous staple height value 72 from a previous surgery, for both thin and thick tissue, are saved or recorded on at least one of the console 52 and the controller 54. Then during a subsequent surgery, the user can interact with a touchpoint 58 to have the previous values 70, 72 (for thin or thick tissue) displayed as a mental reference point for the user. Additionally, the final previous compression value 70 and the final previous staple height value 72 can be linked and saved within a profile for each specific surgeon, such that the final previous compression value 70 and the final previous staple height value 72 are linked to each specific surgeon and their preferences. The aforementioned embodiment ensures that consistent tissue compression and staple height are being achieved during surgeries, ensuring consistent results and successful surgeries.

Additionally or alternatively, referring to FIG. 10B, the console display 56 can show the limits of tissue compression to a user during use of the circular stapler 10, while also including an incremental progress indicator 74 for the compression zone 62 and an incremental progress indicator 76 for the staple height 68. The incremental progress indicator 74 for the compression zone 62 is configured to fill a polymeric shape (in the illustrated example an oval) with a designated color once a preset amount of torque or compression has been surpassed. Each subsequent polymeric shape in the incremental progress indicator 74 continues to be filled as further preset amounts of torque or compression have been surpassed. The incremental progress indicator 76 for the staple height 68 is configured to fill a polymeric shape (in the illustrated example an oval) with a designated color once the staple has been compressed to a certain degree. Each subsequent polymeric shape in the incremental progress indicator 76 continues to be filled as the staple is further compressed passed certain preset height values. In some examples, each of the polymeric shapes can be completely filled when a preset value is reached, or each of the polymeric shapes can be filled incrementally (i.e., gradually) as the preset value is reached.

As illustrated in FIG. 10B, the last polymeric shape has not been filled in yet for the incremental progress indicator 74, indicating to the user that the correct amount of torque or compression has not been achieved to reach the final previous compression value 70. Further, the last polymeric shape has not been filled in yet for the incremental progress indicator 76, indicating to the user that the correct staple height has not been achieved to reach the final previous staple height value 72. This embodiment provides a different visual representation to the user, making it easy for the user to remember and determine that consistent tissue compression and staple height are being achieved during surgeries, ensuring consistent results and successful surgeries. In addition, in some examples, an audible or haptic feedback can be provided to the user upon each of the polymeric shapes of the incremental progress indicators 74, 76 being filled. It is to be understood that in some examples both of the incremental progress indicators 74, 76 may be included. In other examples, only one of the incremental progress indicator 74 or the incremental progress indicator 76 may be included. In further examples, neither of the incremental progress indicators 74, 76 may be included.

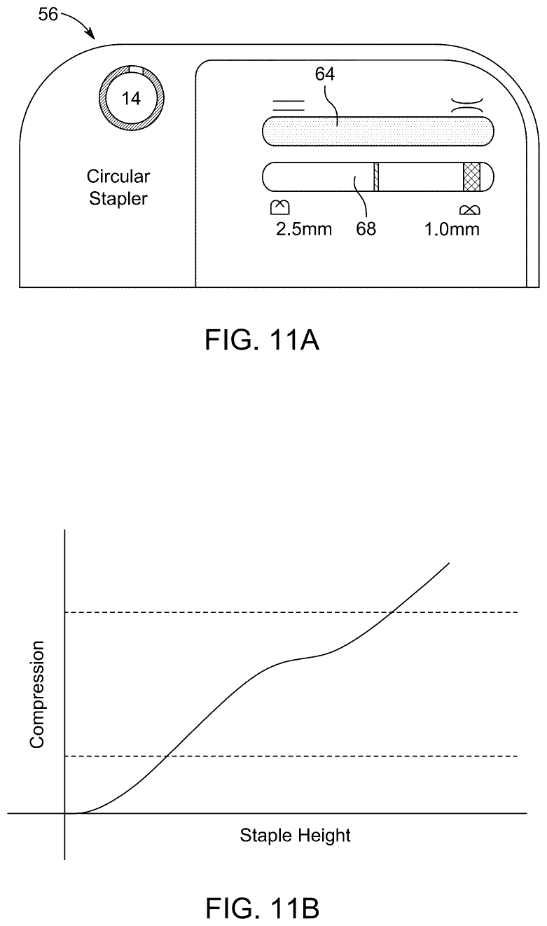

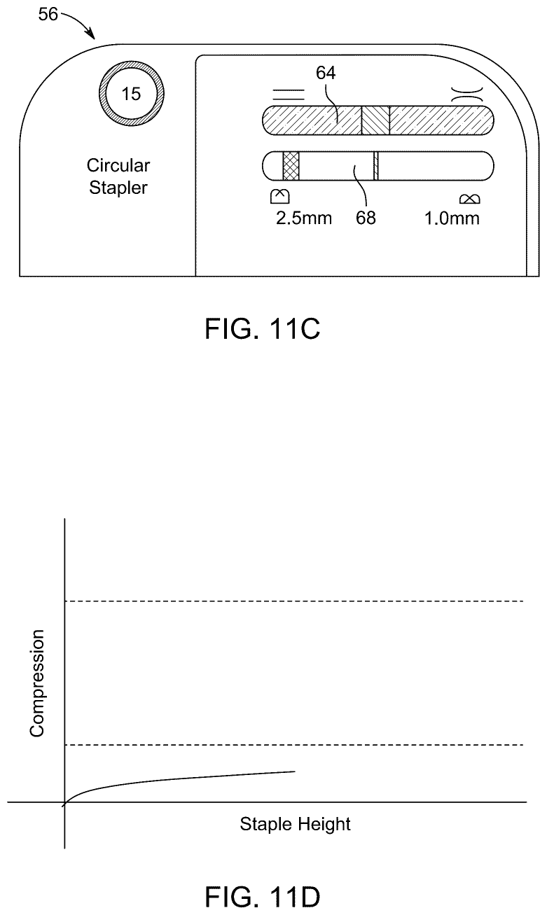

FIG. 11A is another embodiment of the console display 56 that shows limit warnings for compression to a user during use of the circular stapler 10. FIG. 11B is a graphical representation of the limits of compression based on testing data, for the console display 56 of FIG. 11A. FIG. 11C is another embodiment of the console display 56 that shows limit warnings for compression to a user during use of the circular stapler 10. FIG. 11D is a graphical representation of the limits of compression based on testing data, for the console display 56 of FIG. 11C. The embodiment of the console display 56 in FIGS. 11A-11D is configured to provide tissue compression warnings to the user. Specifically, the console display 56 of FIGS. 11A-11D is configured to notify the user if the tissue compression between the anvil 12 and the stapling head 22 is under or over compressed relative to the test values detected during previous testing and calibration of the circular stapler 10.

As shown in FIGS. 11A-11B, if the tissue is compressed beyond an upper compression limit established during previous testing, the compression zone 62 on the console display 56 will flash or otherwise indicate to the user that the tissue is over-compressed. In some examples, the compression zone 62 can flash red to indicate to the user that the tissue is over-compressed. As shown in FIGS. 11C-11D, if the tissue is compressed below a lower compression limit established during previous testing, and the compression is paused for a pre-determined amount of time (e.g. two seconds), the compression zone 62 on the console display 56 will flash or otherwise indicate to the user that the tissue is under-compressed. In some examples, the compression zone 62 can flash yellow to indicate to the user that the tissue is under-compressed. Additionally, or alternatively, at least one of the circular stapler 10, console 52, and controller 54 can produce an audible ping or other distinct sound to indicate to the user that the tissue is either under or over compressed. The console display 56 of FIGS. 11A-11D is configured to notify the user if the tissue compression between the anvil 12 and the stapling head 22 is under or over compressed relative to the test values detected during previous testing and calibration of the circular stapler 10, to ensure proper tissue compression before the cutting and stapling processes.

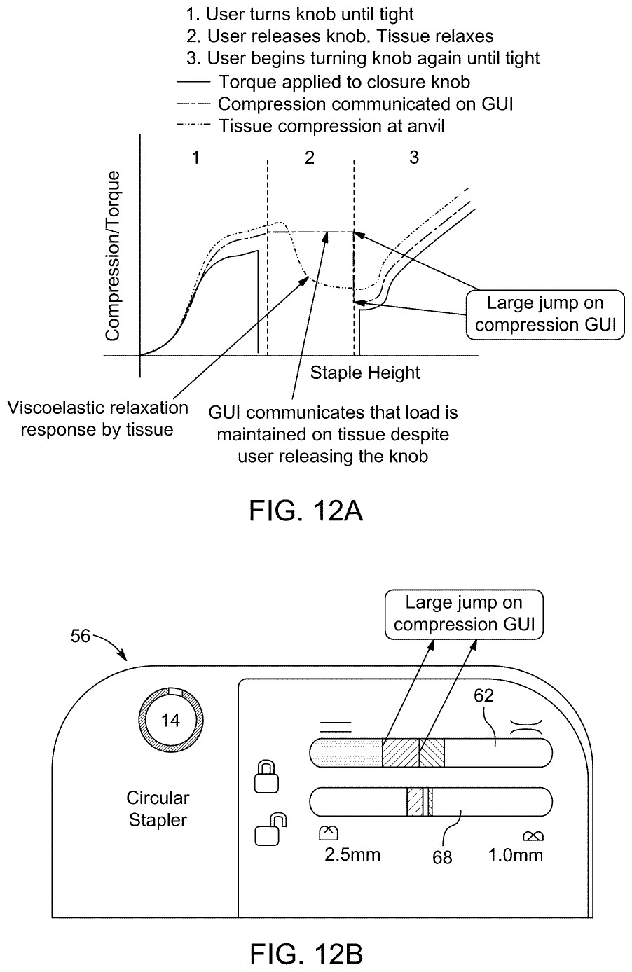

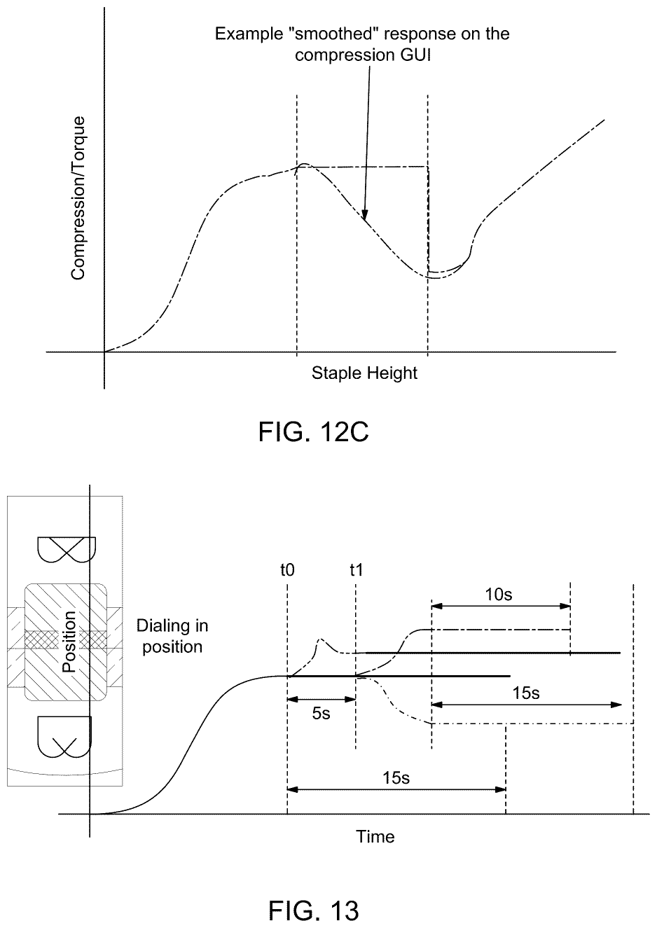

FIG. 12A is a graphical representation of tissue compression/knob torque versus the staple height during compression of the tissue. FIG. 12B is a schematic diagram of the console display 56 that shows the limits of compression to a user during use of the circular stapler 10. FIG. 12C is a graphical representation of an algorithm, within at least one of the console 52 and the controller 54, smoothing the curve of tissue compression/knob torque versus the staple height during compression of the tissue. As illustrated in FIG. 12A, while the user turns the adjustment knob 32 to compress the tissue between the anvil 12 and the stapling head 22 (i.e., zone “1”), the torque applied to the adjustment knob 32 (detected by the torque sensor 44) increases and the compression value communicated to the compression zone 62 of the console display 56 increases. Then when a user releases the adjustment knob 32 and does not continue compressing tissue (i.e., zone “2”), the torque applied to the adjustment knob 32 drops to zero and the tissue compression at the anvil 12 drops due to the viscoelastic relaxation response of the tissue. Then when a user continues tightening the adjustment knob 32 to continue compressing the tissue (i.e., zone “3”), there is a large, sudden jump in compression indicated in the compression zone 62 displayed on the console display 56, which could be confusing to the surgeon.

As such, in the embodiment of the console display 56 in FIGS. 12A-12B, an algorithm is implemented in at least one of the console 52 and the controller 54 that smooths out the compression value displayed in the compression zone 62 on the console display 56. The smoothing of the compression value comprises slightly changing the values that are displayed on the console display 56 compared to the actual values gathered by the sensors (e.g. torque sensors 44, force sensors 46A, 46B, etc.). More specifically, when a user releases the adjustment knob 32 and does not continue compressing the tissue (i.e., zone “2”), the compression value indicated in the compression zone 62 on the console display 56 remains constant (i.e., the load or force is being maintained on the tissue) or only slightly decreases. Then when the user continues tightening the adjustment knob 32 to continue compressing the tissue (i.e., zone “3”), the smoothing algorithm causes the compression value (i.e. the compression bar in the compression zone 62) to perform a “catch-up” to the sensor data by quickly adjusting the compression value without the sudden jump, preventing confusion for the surgeon. The embodiment illustrated in FIGS. 12A-12C is configured to ensure proper tissue compression before the cutting and stapling processes, while preventing confusion for the surgeon by algorithmically smoothing the compression value indicated in the compression zone 62.

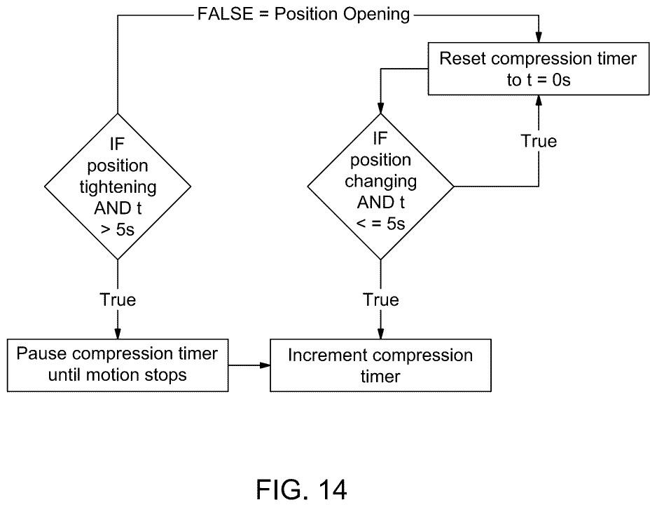

FIG. 13 is a graphical representation of a process for implementing a pre-compression timer according to the present disclosure. FIG. 14 is a schematic representation of the process for implementing a pre-compression timer according to the present disclosure. Currently, instructions for the circular stapler 10, presented by the console 52 or the controller 54, may indicate that the surgeon needs to tighten the circular stapler 10 after pre-compression, but the algorithm tracking the motion of the circular stapler 10 would normally reset the timer in this situation. Therefore, an embodiment of an algorithm for pre-compression time increment, implemented in either the console 52 or the controller 54, accounts for further closure by implementing a “pause” if tightening motion is detected at an initial time threshold. More specifically, the pre-compression timer algorithm can account for further closure of the circular stapler 10 after an initial time threshold (t1, 5 s in example shown in FIG. 13), and then the algorithm can implement a pause in the timer but it does not fully reset the timer for this situation. In some examples, the closure position could be sensed from an encoder position on and coupled to the device.

The circular stapler 10 of the present disclosure alleviates the issues of determining whether the proper tissue compression has been achieved by providing feedback to the surgeon that proper tissue compression has been achieved before firing of the circular stapler 10. Several different embodiments are provided, with each detection system providing an indication to the surgeon that proper tissue compression has been achieved before firing of the circular stapler 10. As will be appreciated by those having skill in the art, the circular stapler 10 of the present disclosure provides many advantages over previously known circular staplers.

Having thus described the present embodiments in detail, it is to be appreciated and will be apparent to those skilled in the art that many physical changes, only a few of which are exemplified in the detailed description of the disclosure, could be made without altering the inventive concepts and principles embodied therein. It is also to be appreciated that numerous embodiments incorporating only part of the preferred embodiment are possible which do not alter, with respect to those parts, the inventive concepts and principles embodied therein.

The present embodiment and optional configurations are therefore to be considered in all respects as exemplary and/or illustrative and not restrictive, the scope of the disclosure being indicated by the appended claims rather than by the foregoing description, and all alternate embodiments and changes to this embodiment which come within the meaning and range of equivalency of said claims are therefore to be embraced therein.

What is claimed is:

1. A method of determining and displaying tissue compression in a circular stapler, the method comprising:
establishing a range of expected compression values based on known tissue properties;
gathering a tissue compression value for tissue compressed between an anvil of the circular stapler and a stapling head of the circular stapler;
comparing the gathered tissue compression value to the range of expected compression values; and
providing an indication that the gathered tissue compression value is within the range of expected compression values.

2. The method according to claim 1, wherein the range of expected compression values is established based on test data indicating a maximum tissue compression and a minimum tissue compression.

3. The method according to claim 2, wherein the maximum tissue compression is a maximum burst pressure of the tissue, and the minimum tissue compression is a minimum burst pressure of the tissue.

4. The method according to claim 1, wherein the known tissue properties comprises tissue thickness.

5. The method according to claim 1, wherein the known tissue properties comprises a position along a gastrointestinal tract.

6. The method according to claim 1, further comprising gathering the tissue compression value by at least one of a torque sensor and a force sensor.

7. The method according to claim 6, wherein the torque sensor is positioned within an adjustment knob of the circular stapler.

8. The method according to claim 1, further comprising comparing the gathered tissue compression value and the range of expected compression values within at least one of a console and a controller communicatively coupled to the circular stapler.

9. The method according to claim 8, wherein the console and the controller are wirelessly communicatively coupled to the circular stapler, and the console and the controller are positioned remotely from the circular stapler.

10. The method according to claim 1, further comprising providing the indication that the gathered tissue compression value is within the range of expected compression values on a display of the circular stapler.

11. The method according to claim 1, further comprising providing the indication that the gathered tissue compression value is within the range of expected compression values on a display of a console that is communicatively coupled to the circular stapler.

12. The method according to claim 1, further comprising providing the indication that the gathered tissue compression value is within the range of expected compression values on a display of a controller that is communicatively coupled to the circular stapler.

13. The method according to claim 1, further comprising providing an indication of over-compression or under-compression on a display of the circular stapler.

14. The method according to claim 13, further comprising providing an indication of staple height on the display of the circular stapler.

15. The method according to claim 1, further comprising providing an indication of over-compression or under-compression on a display of a console that is communicatively coupled to the circular stapler.

16. The method according to claim 15, further comprising providing an indication of staple height on the display of the console.

17. The method according to claim 1, further comprising providing an indication of over-compression or under-compression on a display of a controller that is communicatively coupled to the circular stapler.

18. The method according to claim 17, further comprising providing an indication of staple height on the display of the controller.

19. A circular stapler apparatus comprising:
an anvil;
a stapling head arranged to cooperate with the anvil to compress tissue therebetween;
an adjustment mechanism operable to move the anvil relative to the stapling head to compress the tissue;
a sensor configured to provide an output indicative of a tissue compression value for the tissue compressed between the anvil and the stapling head;
a display; and
a controller in communication with the sensor and the display, the controller comprising at least one processor and memory storing instructions that, when executed by the at least one processor, cause the controller to:
establish a range of expected compression values based on known tissue properties;
determine, based on the output of the sensor, the tissue compression value;
compare the tissue compression value to the range of expected compression values; and
cause the display to present an indication that the tissue compression value is within the range of expected compression values.

20. The circular stapler apparatus of claim 19, wherein the sensor comprises a torque sensor positioned within an adjustment knob of the circular stapler apparatus and configured to provide the output indicative of the tissue compression value based on torque applied to the adjustment knob.
